(19) United States
(12) Patent Application Publication
(10) Pub. No.: US 2025/0288780 A1
(43) Pub. Date: Sep. 18, 2025

(54) MEDICAL DEVICE

(71) Applicant: ASAHI INTECC CO., LTD., Seto-shi (JP)

(72) Inventors: Aoi OKAMURA, Seto-shi (JP); Kenta Tsuge, Seto-shi (JP)

(73) Assignee: ASAHI INTECC CO., LTD., Seto-shi (JP)

(21) Appl. No.: 19/220,904

(22) Filed: May 28, 2025

Related U.S. Application Data
(63) Continuation of application No. PCT/JP2022/044153, filed on Nov. 30, 2022.

(51) Int. Cl. A61M 25/09

(52) U.S. Cl. CPC A61M 25/09; A61M 2025/09083

(57) ABSTRACT

A medical device includes: a core shaft; a first coil body that is arranged so as to surround a distal end portion of the core shaft; a second coil body that is disposed outside the first coil body in a radial direction and has a distal end located between a distal end and a proximal end of the first coil body in an axial direction of the core shaft; and a fixation portion that fixes a vicinity of the distal end of the second coil body and the first coil body, in which, on a portion between an outer periphery and an inner periphery of the second coil body, an axially directional position of a distal end of the fixation portion is located on a position identical to or on a proximal end side with respect to an axially directional position of the distal end of the second coil body.

### CROSS REFERENCE TO RELATED APPLICATIONS

This is a Bypass Continuation Application based on PCT Application PCT/JP2022/044153 filed Nov. 30, 2022. The disclosure of the prior application is hereby incorporated by reference herein in its entirety.

### TECHNICAL FIELD

The disclosed embodiments relate to a medical device such as a guide wire and a catheter.

### BACKGROUND

Guide wires having a plurality of coils that cover an outer periphery of a core shaft have been conventionally known. Patent Literature 1 describes a guide wire having a first coil covering an outer periphery of a core shaft and a second coil covering an outer periphery of the first coil, in which the first coil and the second coil are fixed.

### CITATION LIST

### Patent Literature

Patent Literature 1: JP 2009-511184 W

### SUMMARY

### Technical Problem

Conventional guide wires have had a problem where folding of a guide wire sometimes extends (progresses) towards a proximal end side due to a gradually changed bending rigidity in the axial direction of the guide wire, or a small amount of change in the bending rigidity in the axial direction of the guide wire. Such a problem was not unique to guide wires, but was also common to medical devices such as catheters.

An object of the disclosed embodiments is to prevent a folding of a medical device such as a guide wire from extending toward the proximal end side.

### Solution to Problem

The disclosed embodiments have been made to solve at least a part of the above problems and can be embodied as the following aspects.

(1) An aspect of the disclosed embodiments is a medical device. The medical device includes: a core shaft; a first coil body that is arranged so as to surround a distal end portion of the core shaft; a second coil body that is disposed outside the first coil body in a radial direction and has a distal end located between a distal end and a proximal end of the first coil body in an axial direction of the core shaft; and a fixation portion that fixes a vicinity of the distal end of the second coil body and the first coil body, in which, on a portion between an outer periphery and an inner periphery of the second coil body, an axially directional position of a distal end of the fixation portion is located on a position identical to or on a proximal end side with respect to an axially directional position of the distal end of the second coil body.

According to this configuration, the distal end of the fixation portion is located on a position identical to or on a proximal end side with respect to the distal end of the second coil body, so that the folding of the medical device can be prevented from extending (progressing) toward the proximal end side.

### BRIEF DESCRIPTION OF DRAWINGS

FIG. 1 is an explanatory view illustrating an overall configuration of a medical device according to the first embodiment.

FIG. 2 is an explanatory view illustrating a cross-section taken along line A-A in FIG. 1.

FIG. 3 is an explanatory view illustrating a first fixation portion.

FIG. 4 is an explanatory diagram presenting a bending rigidity of the medical device according to the first embodiment.

FIG. 5 is an explanatory view illustrating a first fixation portion of a conventional medical device.

FIG. 6 is an explanatory diagram illustrating a bending rigidity of the conventional medical device.

FIG. 7 is an explanatory diagram illustrating a method for measuring the bending rigidity.

FIG. 8 is an explanatory view illustrating a first fixation portion of a medical device according to the second embodiment.

FIG. 9 is an explanatory view illustrating a first fixation portion of a medical device according to the third embodiment.

FIG. 10 is an explanatory view illustrating a first fixation portion of a medical device according to the fourth embodiment.

FIG. 11 is an explanatory view illustrating a first fixation portion of a medical device according to the fifth embodiment.

FIG. 12 is an explanatory view illustrating a first fixation portion of a medical device according to the sixth embodiment.

FIG. 13 is an explanatory view illustrating a distal end portion of a medical device according to the seventh embodiment.

### DETAILED DESCRIPTION

### First Embodiment

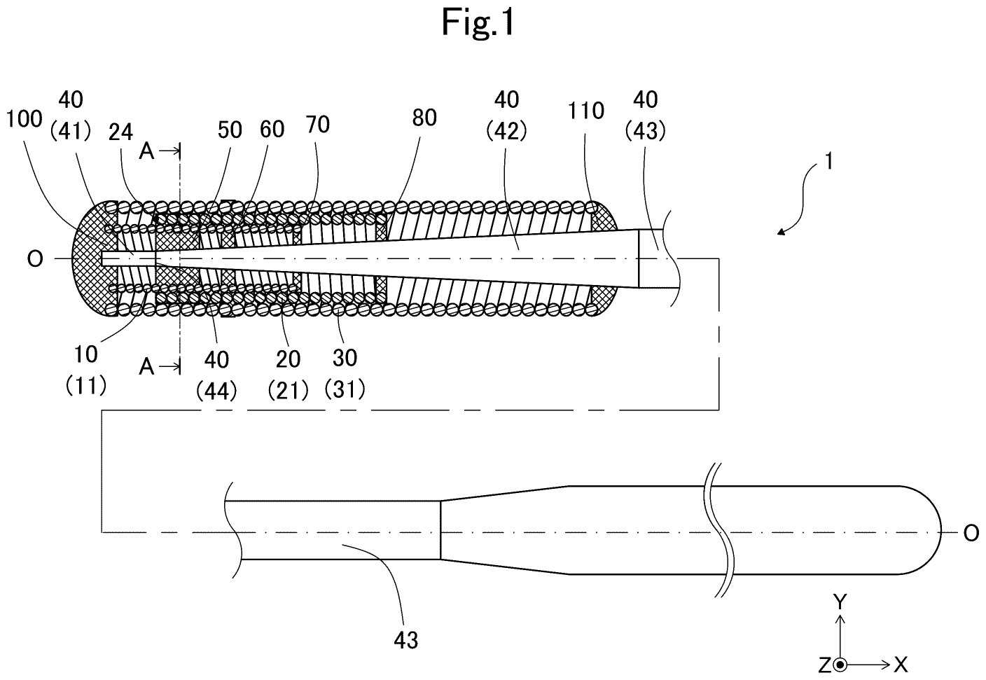

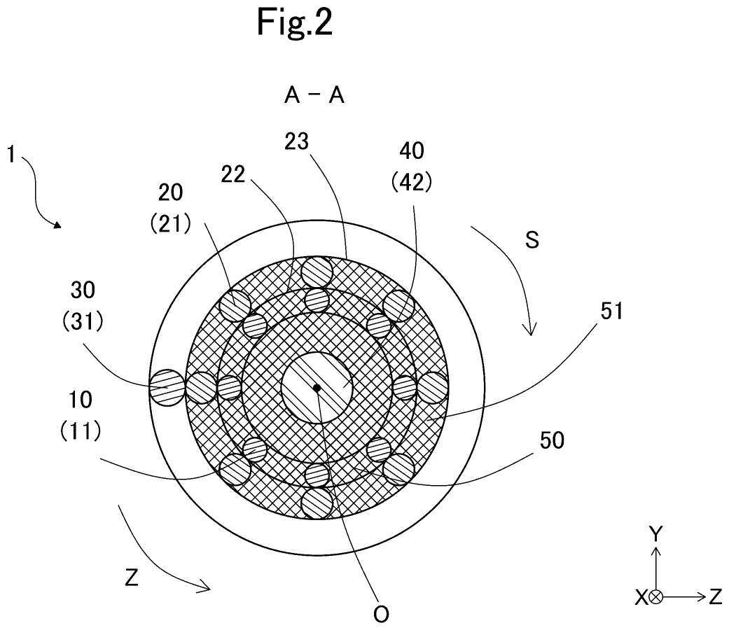

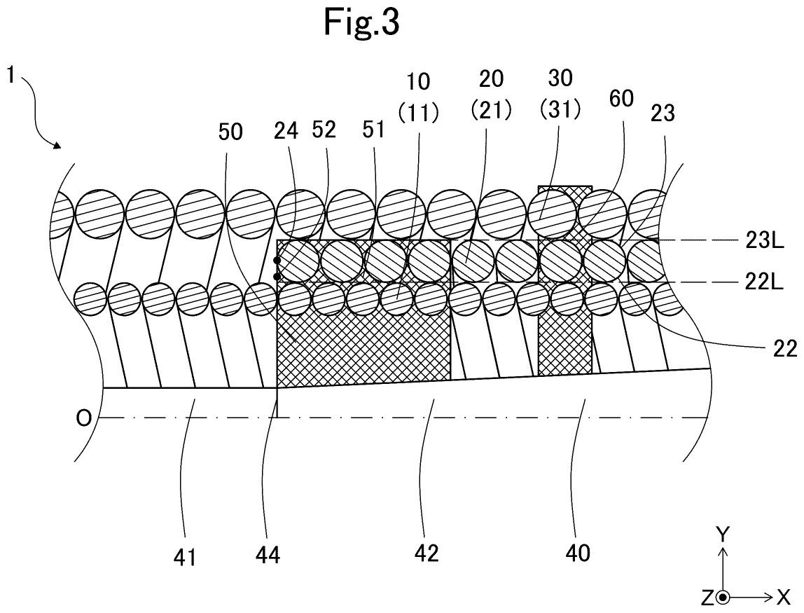

FIG. 1 is an explanatory view illustrating a whole configuration of a medical device 1 according to the first embodiment. FIG. 2 is an explanatory view illustrating a cross-section taken along line A-A in FIG. 1. The medical device 1 according to the first embodiment will be explained with reference to FIG. 1 to FIG. 4. The medical device 1 will be explained e.g. as a guide wire for use in inserting a medical appliance such as a catheter into a blood vessel or the like. The medical device 1 includes a first coil body 10, a second coil body 20, a third coil body 30, a core shaft 40, a first fixation portion 50, a second fixation portion 60, a third fixation portion 70, a fourth fixation portion 80, a distal end-side fixation portion 100, and a proximal end-side fixation portion 110. FIG. 1 and FIG. 3 are longitudinal sectional views illustrating each member constituting the medical device 1, in which the core shaft 40 is illustrated as an outer shape. Although the medical device 1 will be explained as an appliance to be used for blood vessels in the following examples, the medical device 1 can be inserted not only into vascular systems but also into living body lumens such as lymphatic system, biliary system, urinary system, respiratory tract system, digestive system, secreting gland, and reproductive organs.

In FIG. 1, the axis passing through the center of the medical device 1 is represented by an axis line O (dash-dotted line). In the example illustrated in FIG. 1, the axis line O coincides with each axis passing through each center of the first to third coil bodies (10, 20, 30) and the core shaft 40. However, the axis line O may be deviated from each central axis of each constituent described above. In FIG. 1, mutually-orthogonal X, Y, and Z axes are illustrated. The X axis corresponds to an axial direction of the medical device 1, the Y axis corresponds to a height direction of the medical device 1, and the Z axis corresponds to a width direction of the medical device 1. The left side (−X axis direction) in FIG. 1 is referred to as “distal end side” of the medical device 1 and each component. The right side (+X axis direction) in FIG. 1 is referred to as “proximal end side” of the medical device 1 and each component. Among the both ends in the axis direction (X axis direction) of the medical device 1 and each component, one end on the distal end side is referred to as “distal end”, and the other end on the proximal end side is referred to as “proximal end”. The distal end and a vicinity thereof are referred to as “distal end portion”, and the proximal end and a vicinity thereof are referred to as “proximal end portion”. The distal end side is inserted into a living body, and the proximal end side is operated by an operator such as a surgeon. These configurations are common with the figures following FIG. 1.

### Core Shaft 40

As illustrated in FIG. 1, the core shaft 40 has an elongated outer shape extending along the axis line O. The core shaft 40 has a straight portion 41, a tapered portion 42, and a large diameter portion 43 in this order from the distal end to the proximal end.

The straight portion 41 is disposed on the frontmost end side of the core shaft 40. The straight portion 41 has an elongated shape extending coaxially with the axis line O of the medical device 1. The straight portion 41 has an outer diameter substantially constant along the axis line O. The distal end of the straight portion 41 is fixed to the first coil body 10 and the third coil body 30 by the distal end-side fixation portion 100. The tapered portion 42 is connected to the proximal end of the straight portion 41, and the large diameter portion 43 is connected to the proximal end of the tapered portion 42.

The straight portion 41 is a member for making it easy for an operator to shape the distal end portion of the medical device 1 into a curved shape, and also referred to as “ribbon”. The straight portion 41 may have a flattened transverse section for making it easy for an operator to shape the distal end portion into a curved shape. For example, the straight portion 41 may have a transverse sectional shape with the maximum length in the Y axis direction different from the maximum length in the Z axis direction, which is not illustrated in the figure. Specifically, the straight portion 41 may have a rectangular or elliptical transverse sectional shape with the maximum length in the Z axis direction larger than the maximum length in the Y axis direction.

The tapered portion 42 is disposed between the straight portion 41 and the large diameter portion 43. The tapered portion 42 has an almost truncated conical shape with an outer diameter increasing from the distal end side toward the proximal end side in the axial direction of the core shaft 40. The straight portion 41 is connected to a distal end 44 of the tapered portion 42, and the large diameter portion 43 is connected to the proximal end of the tapered portion 42. FIG. 2 illustrates a transverse section of the tapered portion 42. The tapered portion 42 has a circular transverse section.

The large diameter portion 43 is disposed on the rearmost end side of the core shaft 40. The large diameter portion 43 has an almost columnar shape with an outer diameter substantially constant from the proximal end to the distal end. The large diameter portion 43 has an outer diameter equal to the largest diameter of the tapered portion 42. Note that, in the first embodiment, the term “equal” means being substantially equal and allows differences due to production errors and the like. The tapered portion 42 is connected to the distal end of the large diameter portion 43. The proximal end portion of the large diameter portion 43 is held and operated by an operator.

Examples of the material used for the core shaft 40 may include, but are not particularly limited to, a stainless steel (e.g. SUS302, SUS304, and SUS316), a superelastic alloy such as nickel-titanium (Ni—Ti) alloy (also referred to as pseudoelastic alloy), a piano wire, a nickel-chromium alloy, a cobalt alloy, platinum, gold, and tungsten. The straight portion 41, the tapered portion 42, and the large diameter portion 43 may be made of a same material or different materials.

### First Coil Body 10

The first coil body 10, the second coil body 20, and the third coil body 30 are disposed on the outer periphery of the core shaft 40 in this order from the inner side to the outer side of the medical device 1 in the radial direction. The first coil body 10 is formed from eight wires 11 spirally wound in the axial direction of the medical device 1. The first coil body 10 is arranged so as to surround the distal end portion of the core shaft 40. Specifically, the first coil body 10 is arranged so as to surround the straight portion 41 and a part of the tapered portion 42 on the distal end side in the core shaft 40. The distal end of the first coil body 10 is fixed to the core shaft 40 and the third coil body 30 by the distal end-side fixation portion 100. The proximal end of the first coil body 10 is fixed to the core shaft 40 by the third fixation portion 70.

The first coil body 10 is wound in a first winding direction S as illustrated in FIG. 2. The first winding direction S refers to a clockwise rotational direction around the axis line O passing through the center of the medical device 1 at a viewpoint for viewing the proximal end side from the distal end side of the medical device 1 (in other words, in the +X axis direction).

### Second Coil Body 20

The second coil body 20 is formed from eight wires 21 spirally wound in the axial direction of the medical device 1. The second coil body 20 is disposed outside the first coil body 10 in the radial direction of the medical device 1, to surround a part of the tapered portion 42 and a part of the first coil body 10. A distal end 24 of the second coil body 20 is located between the distal end and the proximal end of the first coil body 10 in the axial direction of the medical device 1. The distal end 24 of the second coil body 20 is fixed to the first coil body 10 and the core shaft 40 by the first fixation portion 50. The proximal end of the second coil body 20 is located on the proximal end side with respect to the proximal end of the first coil body 10 in the axial direction of the medical device 1. An intermediate portion of the second coil body 20 located between the distal end 24 and the proximal end of the second coil body 20 is fixed to the first coil body 10, the third coil body 30, and the core shaft 40 by the second fixation portion 60. The proximal end of the second coil body 20 is fixed to the core shaft 40 by the fourth fixation portion 80. The second coil body 20 is wound in the first winding direction S as illustrated in FIG. 2. That means, the first coil body 10 and the second coil body 20 are wound in the same direction.

As illustrated in FIG. 2, the second coil body 20 has an inner periphery 22 and an outer periphery 23. The inner periphery 22 is an almost hollow-cylindrical portion formed by aligning the inner sides of the plurality of the spirally wound wires 21. The outer periphery 23 is an almost hollow-cylindrical portion formed by aligning the outer sides of the plurality of the spirally wound wires 21. In the first embodiment, the outer periphery of the first coil body 10 and the inner periphery 22 of the second coil body 20 are in contact with each other, the outer periphery 23 of the second coil body 20 and the inner periphery of the third coil body 30 are in contact with each other. However, the outer periphery of the first coil body 10 and the inner periphery 22 of the second coil body 20 may be away from each other, and the outer periphery 23 of the second coil body 20 and the inner periphery of the third coil body 30 may be away from each other.

The bending rigidity of the second coil body 20 is higher than the bending rigidity of the first coil body 10. The method for measuring the bending rigidity is described later.

### Third Coil Body 30

The third coil body 30 is formed from one wire 31 spirally wound in the axial direction of the medical device 1. The third coil body 30 is disposed outside the second coil body 20 in the radial direction of the medical device 1, to surround a part of the core shaft 40 (in the first embodiment, the straight portion 41 and a part of the tapered portion 42 on the distal end side), the first coil body 10, and the second coil body 20. The distal end of the third coil body 30 is located at the same position as of the distal end of the first coil body 10 in the axial direction of the medical device 1. The distal end of the third coil body 30 is fixed to the core shaft 40 and the first coil body 10 by the distal end-side fixation portion 100. The proximal end of the third coil body 30 is located on the proximal end side with respect to the proximal end of the second coil body 20 in the axial direction of the medical device 1. The proximal end of the third coil body 30 is fixed to the core shaft 40 by the proximal end-side fixation portion 110.

The third coil body 30 is wound in a second winding direction Z as illustrated in FIG. 2. The second winding direction Z refers to a counter-clockwise rotational direction around the axis line O passing through the center of the medical device 1 at a viewpoint for viewing the proximal end side from the distal end side of the medical device 1 (in other words, in the +X axis direction).

For the first to third coil bodies (10, 20, 30), any configuration can be adopted, and for example, the number of the wires (11, 21, 31) constituting the first to third coil bodies (10, 20, 30) can be arbitrarily determined. The first coil body 10 and the second coil body 20 are not limited to a multi-thread coil, but also may be a single thread coil formed by winding one wire in a single thread manner, a single thread twisted wire coil formed by winding, in a single thread manner, a twisted wire with a plurality of wires twisted, or a multi-thread twisted wire coil formed by winding, in a multiple thread manner, each of a plurality of twisted wires with a plurality of wires twisted. Alternatively, the first to third coil bodies (10, 20, 30) may be an almost cylindrical tube body.

Examples of the materials for the first to the third coil bodies (10, 20, 30) include, but are not limited to, a stainless steel alloy such as SUS304 and SUS316, a superelastic alloy such as NiTi alloy, a piano wire, a nickel-chromium alloy, a radiolucent alloy such as a cobalt alloy, gold, platinum, tungsten, and a radiopaque alloy such as an alloy containing these elements (e.g. a platinum-nickel alloy). The first to third coil bodies (10, 20, 30) may be made of a same material or may be made of different materials.

### Fixation Portion

The core shaft 40, the first coil body 10, the second coil body 20, and the third coil body 30, which constitute the medical device 1, are fixed by the plurality of fixation portions. The medical device 1 has a distal end-side fixation portion 100, a first fixation portion 50, a second fixation portion 60, a third fixation portion 70, a fourth fixation portion 80, and a proximal end-side fixation portion 110 in this order from the distal end side of the medical device 1.

The distal end-side fixation portion 100 is disposed on the distal end of the third coil body 30 to fix the distal end of the third coil body 30, the distal end of the core shaft 40, and the distal end of the first coil body 10. The proximal end-side fixation portion 110 is disposed on the proximal end of the third coil body 30 to fix the proximal end of the third coil body 30 and a part of the tapered portion 42. The first fixation portion 50 is disposed on the distal end 24 of the second coil body 20 to fix the distal end 24 of the second coil body 20 and a part of the first coil body 10. The second fixation portion 60 is disposed on the intermediate portion of the second coil body 20 to fix the first coil body 10, the second coil body 20, the third coil body 30, and the core shaft 40. The third fixation portion 70 is disposed on the proximal end of the first coil body 10 to fix the proximal end of the first coil body 10 and a part of the tapered portion 42. The fourth fixation portion 80 is disposed on the proximal end of the second coil body 20 to fix the proximal end of the second coil body 20 and a part of the tapered portion 42.

As illustrated in FIG. 2, the first fixation portion 50 is formed between the outer periphery of the core shaft 40 and the outer periphery 23 of the second coil body 20. A specific portion 51 that is a part of the first fixation portion 50 is formed between the inner periphery 22 and the outer periphery 23 of the second fixation portion 60. The first fixation portion 50 is described later in detail.

The distal end-side fixation portion 100, the proximal end-side fixation portion 110, the first fixation portion 50, the second fixation portion 60, the third fixation portion 70, and the fourth fixation portion 80 can be made of any bonding agent, e.g. a metal solder such as silver braze, gold braze, zinc, Sn—Ag alloy, and Au—Sn alloy, or an adhesive such as an epoxy adhesive. The distal end-side fixation portion 100, the proximal end-side fixation portion 110, the first fixation portion 50, the second fixation portion 60, the third fixation portion 70, and the fourth fixation portion 80 may be made of a same bonding agent, or may be made of different bonding agents. The distal end-side fixation portion 100, the proximal end-side fixation portion 110, the first fixation portion 50, the second fixation portion 60, the third fixation portion 70, and the fourth fixation portion 80 may be formed by laser welding.

### Detailed Description of First Fixation Portion 50

FIG. 3 is an explanatory view illustrating the first fixation portion 50. The first fixation portion 50 fixes the vicinity of the distal end 24 of the second coil body 20, the core shaft 40, and the first coil body 10. The “vicinity of the distal end 24 of the second coil body 20” means the distal end portion of the second coil body 20, which extends from the distal end 24 toward the proximal end side of the second coil body 20 (in the +X axis direction) by a predetermined distance. The “predetermined distance” means e.g. a distance of an axially directional distance of the second coil body 20 (distance in +X axis direction) equate to one to about ten wires 21 on the longitudinal section of the second coil body 20 as illustrated in FIG. 3.

In this embodiment, a portion of the first fixation portion 50, located between the inner periphery 22 and the outer periphery 23 of the second coil body 20 is referred to as “specific portion 51”. More specifically, the specific portion 51 is located between a radially directional innermost portion of the inner periphery 22 of the second coil body 20 and a radially directional outermost portion of the outer periphery 23 of the second coil body 20, in the first fixation portion 50. In FIG. 3, a virtual line 22L and a virtual line 23L are illustrated. The virtual line 22L is a line that virtually connects radially directional innermost portions of the inner periphery 22. The virtual line 23L is a line that virtually connects radially directional outermost portions of the outer periphery 23. The phrase “disposed between the inner periphery 22 and the outer periphery 23 of the second coil body 20” can be rephrased as “disposed between the virtual line 22L and the virtual line 23L”. The specific portion 51 is formed along spaces between wires 21 adjacent to each other in the axial direction of the second coil body 20 and along the outer periphery of the wire 21. The frontmost portion of the specific portion 51 is referred to as “distal end 52”. In this case, an axially directional position of the distal end 52 of the specific portion 51 is the same as an axially directional position of the distal end 24 of the second coil body 20, or is located on the proximal end side with respect to the axially directional position of the distal end 24 of the second coil body 20. Thereby, as described later, a portion where the bending rigidity of the medical device 1 significantly changes can be formed on the distal end 24 of the second coil body 20 to prevent the folding of the medical device 1 on the distal end side from extending (progressing) toward the proximal end side with respect to the distal end 24 of the second coil body 20. The axially directional position of the distal end 52 of the specific portion 51 is the same as an axially directional position of the distal end 44 of the tapered portion 42, or is located on the proximal end side with respect to the axially directional position of the distal end 44 of the tapered portion 42. Thereby, as described later, a portion where the bending rigidity of the medical device 1 significantly changes can be formed on the distal end 24 of the second coil body 20 to prevent the folding of the medical device 1 on the distal end side from extending (progressing) toward the proximal end side with respect to the distal end 24 of the second coil body 20.

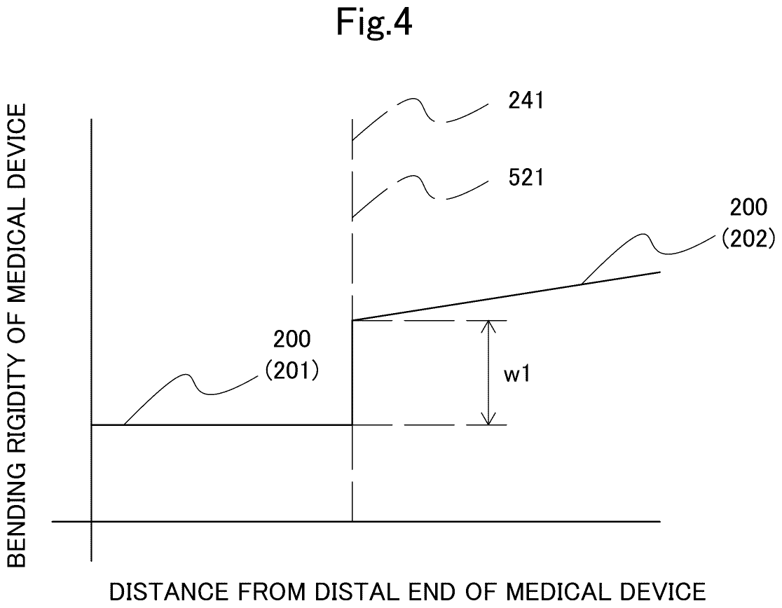

FIG. 4 is an explanatory diagram presenting a bending rigidity 200 of the medical device 1 in the axial direction of the medical device 1. In FIG. 4, the abscissa represents a distance from the distal end of the medical device 1, and the ordinate represents the bending rigidity 200 of the medical device 1 at the position at the distance on the abscissa. An “axially directional position 241” in FIG. 4 indicates the axially directional position of the distal end 24 of the second coil body 20. An “axially directional position 521” in FIG. 4 indicates the axially directional position of the distal end 52 of the specific portion 51. In this figure, the axially directional positions 241 and 521 are located at the same position in the axial direction of the medical device 1. Regarding the bending rigidity 200, a bending rigidity 202 on the proximal end side rapidly increases from the axially directional position 241 as a boundary, compared to a bending rigidity 201 on the distal end side. The bending rigidity 202 on the proximal end side with respect to the axially directional position 241 is higher than the bending rigidity 201 on the distal end side with respect to the axially directional position 241 by an increase width w1. The increase width w1 of the bending rigidity 200 is generated when the bending rigidity of the second coil body 20 and the bending rigidity of the specific portion 51 are added to the bending rigidity 201 of the medical device 1. The bending rigidity 202 on the proximal end side gradually increases toward the proximal end side as the outer diameter of the tapered portion 42 of the core shaft 40 increases toward the proximal end side.

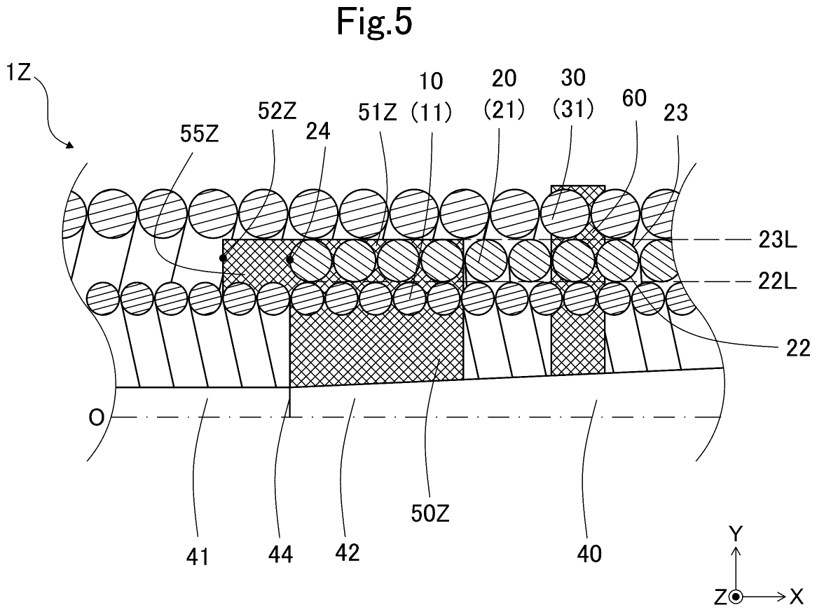

FIG. 5 is an explanatory longitudinal sectional view illustrating a conventional medical device 1Z. Among the members constituting the medical device 1Z, the members excluding a first fixation portion 50Z are common to the members constituting the medical device 1 according to the first embodiment, and therefore their descriptions are omitted. A specific portion 51Z of the medical device 1Z is different from the specific portion 51 of the medical device 1 in that the distal end 52Z is located on the distal end side with respect to the distal end 24 of the second coil body 20. A part of the specific portion 51, formed on the distal end side with respect to the distal end 24 of the second coil body 20 is referred to as “protruding portion 55Z”. The protruding portion 55Z is formed so as to fill the space between the outer periphery of the first coil body 10 and the inner periphery of the third coil body 30 on the distal end side with respect to the distal end 24 of the second coil body 20. On the other hand, the specific portion 51Z excluding the protruding portion 55Z is formed along the outer periphery of the wire 21 of the second coil body 20. Thereby, a volume of the protruding portion 55Z is larger than of the specific portion 51Z excluding the protruding portion 55Z. The bending rigidity of the protruding portion 55Z is also higher than of the specific portion 51Z excluding the protruding portion 55Z.

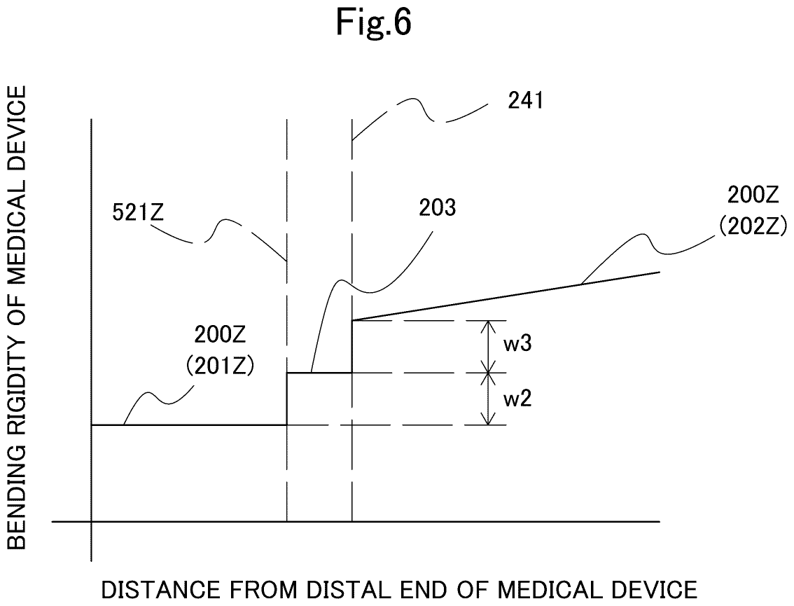

FIG. 6 is an explanatory diagram presenting a bending rigidity 200Z of the medical device 1Z in the axial direction of the medical device 1Z. The “axially directional position 241” in FIG. 6 indicates the axially directional position of the distal end 24 of the second coil body 20. An “axially directional position 521Z” in FIG. 6 indicates the axially directional position of the distal end 52Z of the specific portion 51. In this figure, the axially directional position 241 and the axially directional position 521Z are located at different positions in the axial direction of the medical device 1. The protruding portion 55Z is located between the axially directional position 241 and the axially directional position 521Z. As described above, the axially directional position 521Z of the distal end 52 of the first fixation portion 50Z (axially directional position of the distal end of the protruding portion 55Z) is located on the distal end side with respect to the axially directional position 241 of the distal end 24 of the second coil body 20. Thereby, the bending rigidity 200Z increases on the distal end side with respect to the axially directional position 241 of the distal end 24 of the second coil body 20. Specifically, a bending rigidity 203 on the proximal end side with respect to the axially directional position 521Z is higher than a bending rigidity 201Z on the distal end side of the axially directional position 521Z by an amount (increase width w2) of the bending rigidity of the protruding portion 55Z added to the bending rigidity 200Z. On the other hand, the amount of increase in the bending rigidity 200Z from the axially directional position 241 as a boundary is smaller than the increase width w1 in the medical device 1 (FIG. 4). Specifically, the difference between the increase amount of the bending rigidity 202Z on the proximal end side with respect to the axially directional position 241 and the increase amount of the bending rigidity 203 on the distal end side with respect to the axially directional position 241 is only the increase amount (increase width w3) resulting from the bending rigidity of the second coil body 20. That means, in the medical device 1 according to the first embodiment presented in FIG. 4, the bending rigidity 200 rapidly increases from the distal end 24 of the second coil body 20 as a boundary, whereas in the conventional medical device 1Z, the bending rigidity 200Z gradually increases around the distal end 24 of the second coil body 20. Thus, in the conventional medical device 1Z, the rigidity of the medical device 1 does not significantly change on the distal end 24 of the second coil body 20, and therefore there is a possibility that the folding of the medical device 1 on the distal end side extends (progresses) toward the proximal end side with respect to the distal end 24 of the second coil body 20.

### Method for Measuring Bending Rigidity

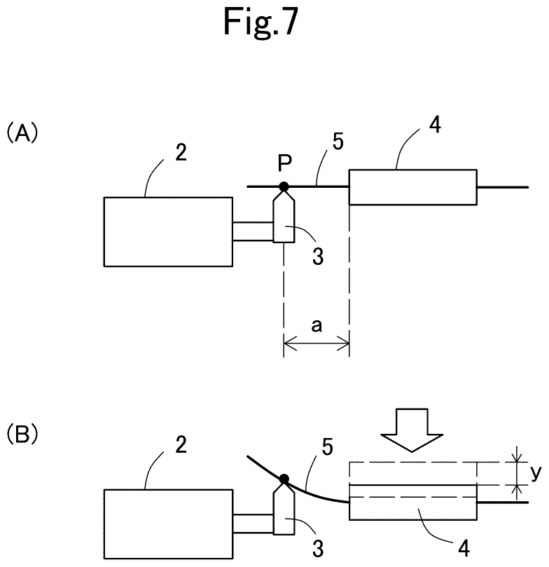

FIG. 7 is an explanatory diagrams illustrating a method for measuring the bending rigidity. In the first embodiment, the bending rigidity is measured by a cantilever test illustrated in FIG. 7. Specifically, as illustrated in FIG. 7A, a measurement object 5 is supported by a gripping tool 4. A load sensor 3 of a measuring device 2 is disposed on and brought into contact with an arbitrary position of the measurement object 5 protruding from the gripping tool 4. In this case, an interval between a contact point P where the measurement object 5 is in contact with the load sensor 3 and an end face of the gripping tool 4 (end face on the side facing the measuring device 2) is defined as a predetermined interval a. In this state, as illustrated in FIG. 7B, the gripping tool 4 is lowered by a predetermined distance in the direction of the arrow in the figure, with the contact point P as a power point. The measurement value obtained from the load sensor 3 at the time when the movement amount of the gripping tool 4 reaches a predetermined displacement amount y is defined as the bending rigidity of the measurement object 5.

In the medical device 1 according to the first embodiment described above, the axially directional position of the distal end 52 of the specific portion 51 is located on the same position as the axially directional position of the distal end 24 of the second coil body 20. As a result, as presented in FIG. 4, the bending rigidity 200 of the medical device 1 rapidly increases from the distal end 24 of the second coil body 20 as a boundary, and the folding of the medical device 1 can be prevented from extending toward the proximal end side with respect to the distal end 24 of the second coil body 20.

For example, in the conventional medical device 1Z described in FIG. 5 and FIG. 6, the distal end 52Z of the specific portion 51Z is disposed on the distal end side with respect to the distal end 24 of the second coil body 20, so that the protruding portion 55Z is formed. Thereby, as presented in FIG. 6, in the axial direction of the medical device 1Z, the amount of the increase in the bending rigidity 200Z from the bending rigidity on the distal end side (201Z) to the bending rigidity on the proximal end side (202Z) with the distal end 24 of the second coil body 20 as a boundary is smaller than that of the medical device 1. In the guide wire 1Z, since the bending rigidity 200Z gradually changes around the distal end 24 of the second coil body 20, it is difficult to prevent the folding of the medical device 1Z on the distal end side from extending toward the proximal end side with respect to the distal end 24 of the second coil body 20. On the other hand, since the difference between the bending rigidity 201 on the distal end side with respect to the distal end 24 of the second coil body 20 and the bending rigidity 202 on the proximal end side is large in the medical device 1, the folding of the medical device 1 on the distal end side can be prevented from extending toward the proximal end side with respect to the distal end 24 of the second coil body 20. In the medical device 1 according to the first embodiment, the axially directional position of the distal end 52 of the specific portion 51 is identical to or on a proximal end side with respect to the axially directional position of the distal end 44 of the tapered portion 42. Thereby, a portion on the distal end 24 of the second coil body 20, where the bending rigidity of the medical device 1 significantly changes, is formed on the proximal end side with respect to the distal end 44 of the tapered portion 42 having a bending rigidity higher than of the straight portion 41, in the core shaft 40. Since the higher a bending rigidity of a portion is, the more likely folding of the portion is suppressed, a portion of the medical device 1 where the bending rigidity significantly changes is formed on a portion of the core shaft 40 where the bending rigidity is relatively high, so that the folding of the medical device 1 on the distal end side can be prevented from extending (progressing) toward the proximal end side with respect to the distal end 24 of the second coil body 20.

The medical device 1 includes the second fixation portion 60 that fixes the core shaft 40, the first coil body 10, the second coil body 20, the third coil body 30, on the proximal end side with respect to the first fixation portion 50. This improves the torquability.

The first coil body 10 and the second coil body 20 are wound in the first winding direction S, and the third coil body 30 is wound in the second winding direction Z. That means, any two of the first coil body 10, the second coil body 20, and the third coil body 30 are wound in the first winding direction S, and the remaining coil body is wound in the second winding direction Z. The coil bodies are each wound in different directions so that the torquability can be improved regardless of the rotational direction of the medical device 1.

The bending rigidity of the second coil body 20 is higher than the bending rigidity of the first coil body 10. This makes it more reliable to prevent the folding of the medical device 1 from extending toward the proximal end side with respect to the distal end 24 of the second coil body 20.

The distal end 52 of the specific portion 51 of the first fixation portion 50 is disposed on the same position as of the distal end 44 of the tapered portion 42 of the core shaft 40. Thereby, the bending rigidity 202 on the proximal end side with respect to the distal end 24 of the second coil body 20 can be further increased to more reliably prevent the folding of the medical device 1 from extending toward the proximal end side with respect to the distal end 24 of the second coil body 20.

### Second Embodiment

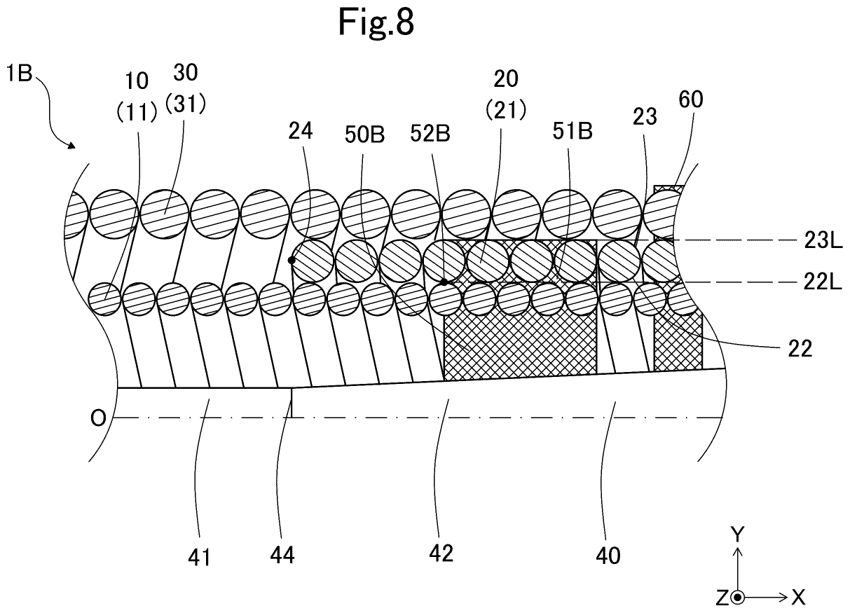

FIG. 8 is an explanatory view illustrating a first fixation portion 50B of a medical device 1B according to the second embodiment. The medical device 1B differs from the medical device 1 according to the first embodiment in that a distal end 52B of the first fixation portion 50B is disposed on the proximal end side with respect to the distal end 24 of the second coil body 20. The descriptions of the components in the medical device 1B, which are common with the components in the medical device 1 according to the first embodiment are omitted.

A bending rigidity of the medical device 1B on the proximal end side with respect to the axially directional position of the distal end 24 of the second coil body 20 rapidly increases compared to the bending rigidity of the medical device 1B on the distal end side, because the medical device 1B on the proximal end side is disposed on the proximal end side with respect to the distal end 24 of the distal end 20 of the second coil body 20. Thereby, the folding of the medical device 1B can be prevented from extending toward the proximal end side with respect to the distal end 24 of the second coil body 20.

### Third Embodiment

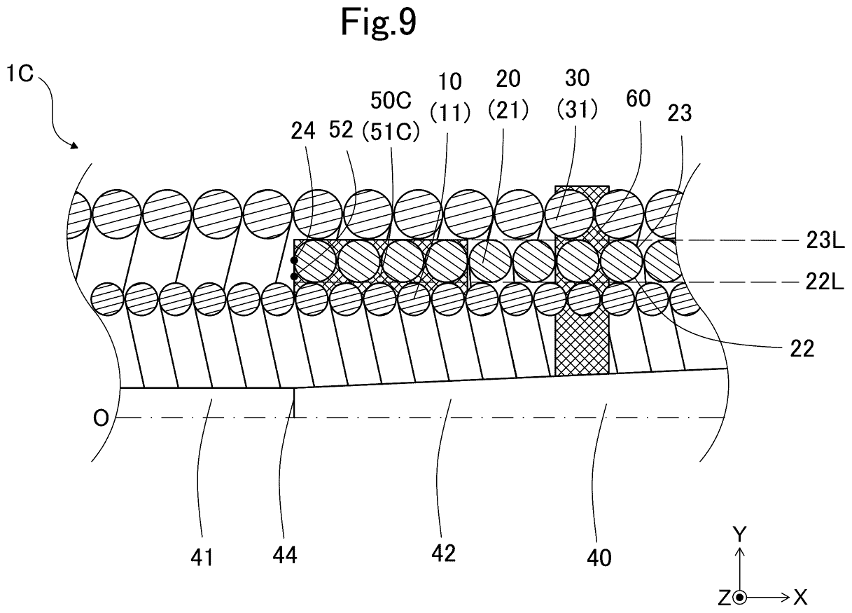

FIG. 9 is an explanatory view illustrating a first fixation portion 50C of a medical device 1C according to the third embodiment. The medical device 1C differs from the medical device 1 according to the first embodiment in that the first fixation portion 50C is not in contact with the core shaft 40 and fixes only the first coil body 10 and the second coil body 20. The descriptions of the components in the medical device 1C, which are common with the components in the medical device 1 according to the first embodiment are omitted.

Also in the medical device 1C according to the third embodiment, a bending rigidity of the medical device 1C on the proximal end side with respect to the axially directional position of the distal end 24 of the second coil body 20 rapidly increases compared to the bending rigidity of the medical device 1C on the distal end side. Thereby, the folding of the medical device 1C can be prevented from extending toward the proximal end side with respect to the distal end 24 of the second coil body 20.

### Fourth Embodiment

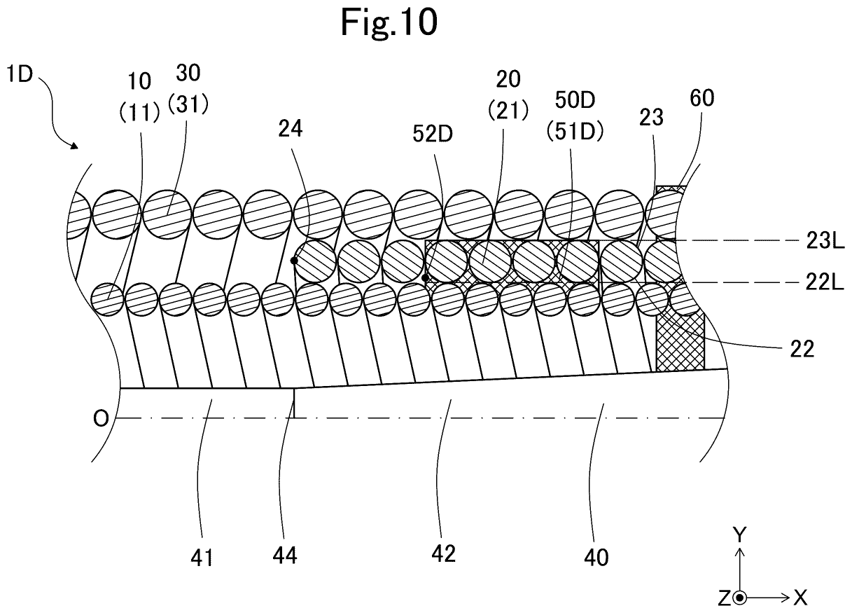

FIG. 10 is an explanatory enlarged view illustrating a first fixation portion 50D of a medical device 1D according to the fourth embodiment. The medical device 1D differs from the medical device 1C according to the third embodiment in that the first fixation portion 50D is disposed on the proximal end side with respect to the distal end 24 of the second coil body 20. The descriptions of the components in the medical device 1D, which are common with the components in the medical device 1C according to the third embodiment are omitted.

Also in the medical device 1D according to the fourth embodiment, a bending rigidity of the medical device 1D on the proximal end side with respect to the axially directional position of the distal end 24 of the second coil body 20 rapidly increases compared to the bending rigidity of the medical device 1D on the distal end side. Thereby, the folding of the medical device 1D can be prevented from extending toward the proximal end side with respect to the distal end 24 of the second coil body 20.

### Fifth Embodiment

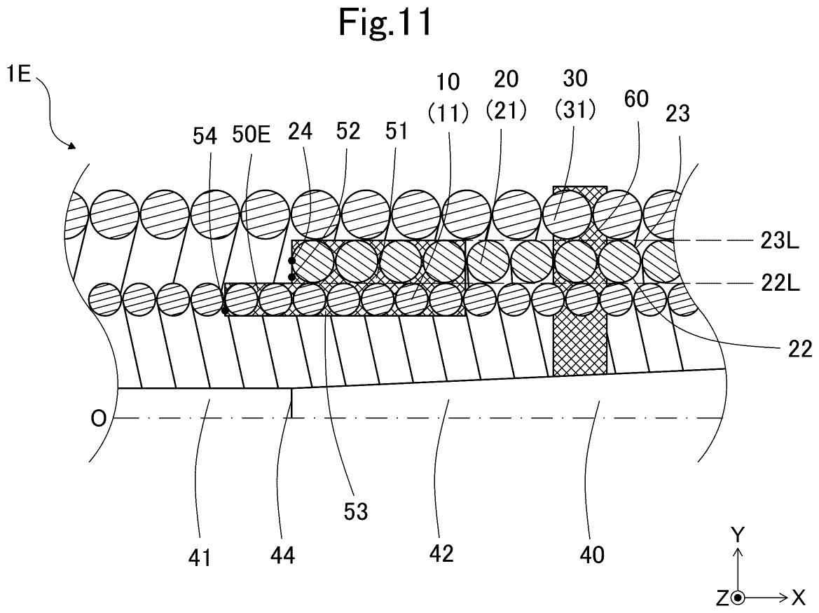

FIG. 11 is an explanatory view illustrating a first fixation portion 50E of a medical device 1E according to the fifth embodiment. The medical device 1E differs from the medical device 1 according to the first embodiment in that a distal end 54 of a portion (hereafter, referred to as a specific portion 53) of the first fixation portion 50E, which is located between the outer and inner peripheries of the first coil body 10, is disposed on the distal end side with respect to the distal end 24 of the second coil body 20. The descriptions of the components in the medical device 1E, which are common with the components in the medical device 1 according to the first embodiment are omitted.

As described above, in this fifth embodiment, the distal end 54 of the specific portion 53 is disposed on the distal end side with respect to the distal end 24 of the second coil body 20. Thereby, a bending rigidity of the medical device 1E on the distal end side with respect to the distal end 24 of the second coil body 20 increases by the amount of the bending rigidity of the specific portion 53. However, since the specific portion 53 is formed merely in a thin form along the outer periphery of the wire 11 of the first coil body 10, the amount of increase in the bending rigidity of the medical device 1E is small. Thus, also in the medical device 1E, the bending rigidity of the medical device 1E on the proximal end side with respect to the axially directional position of the distal end 24 of the second coil body 20 rapidly increases compared to the bending rigidity of the medical device 1E on the distal end side. As a result, the folding of the medical device 1E can be prevented from extending toward the proximal end side with respect to the distal end 24 of the second coil body 20.

### Sixth Embodiment

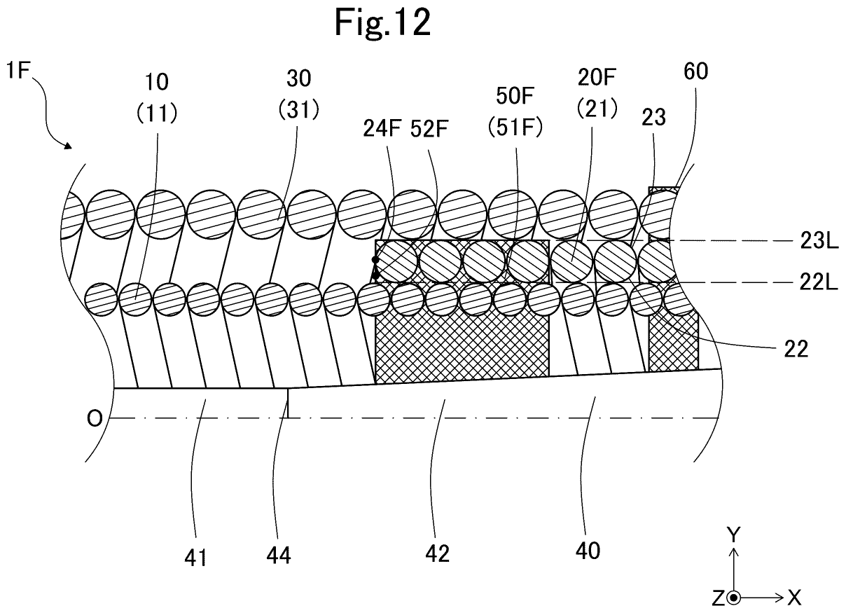

FIG. 12 is an explanatory view illustrating a first fixation portion 50F of a medical device 1F according to the sixth embodiment. The medical device 1F differs from the medical device 1 according to the first embodiment in that a distal end 24F of a second coil body 20F and a distal end 52F of the first fixation portion 50F are disposed on the proximal end side with respect to the distal end 44 of the tapered portion 42. The descriptions of the components in the medical device 1F, which are common with the components in the medical device 1 according to the first embodiment are omitted.

Also in the medical device 1F according to the sixth embodiment, a bending rigidity of the medical device 1F on the proximal end side with respect to the axially directional position of the distal end 24F of the second coil body 20F rapidly increases compared to the bending rigidity of the medical device 1F on the distal end side. Thereby, the folding of the medical device 1F can be prevented from extending toward the proximal end side with respect to the distal end 24F of the second coil body 20F.

### Seventh Embodiment

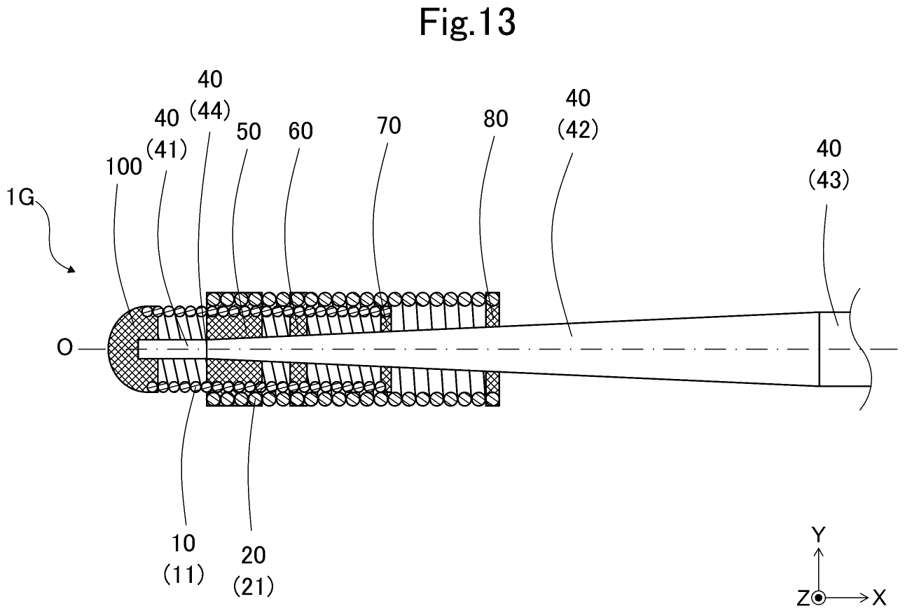

FIG. 13 is an explanatory view illustrating a distal end portion of a medical device 1G according to the seventh embodiment. The medical device 1G differs from the medical device 1 according to the first embodiment in that the medical device 1G does not have the third coil body 30. The descriptions of the components in the medical device 1G, which are common with the components in the medical device 1 according to the first embodiment are omitted.

Also in the medical device 1G according to the seventh embodiment, a bending rigidity of the medical device 1G on the proximal end side with respect to the axially directional position of the distal end 24 of the second coil body 20 rapidly increases compared to the bending rigidity of the medical device 1G on the distal end side. Thereby, the folding of the medical device 1G can be prevented from extending toward the proximal end side with respect to the distal end 24 of the second coil body 20.

### Modification

The disclosed embodiments are not limited to the above-described embodiments and may be implemented in various modes without departing from the gist thereof, and for example, the following modifications are also possible.

### Modification 1

The axially directional lengths of the straight portion 41, the tapered portion 42, and the large diameter portion 43 can be determined as appropriate. For example, in the first and seventh embodiments, the axially directional length of the straight portion 41 is shorter than the axially directional length of the tapered portion 42. However, the axially directional length of the straight portion 41 may be larger than the axially directional length of the tapered portion 42. The transverse sectional shapes of the straight portion 41, the tapered portion 42, and the large diameter portion 43 can be determined as appropriate. For example, in the first embodiment, the transverse sectional shapes of the straight portion 41, the tapered portion 42, and the large diameter portion 43 are circular. However, the transverse sectional shapes of the straight portion 41, the tapered portion 42, and the large diameter portion 43 may be an elliptical shape, or a polygonal shape such as triangular or rectangular shapes.

### Modification 2

The medical devices (1, 1B, 1C, 1D, 1E, IF, 1G) according to the first to seventh embodiments may further have a resin coating film on the outer periphery of the third coil body 30 or the second coil body (20, 20F).

### Modification 3

The second fixation portion 60 may be configured to fix the first coil body 10, the second coil body (20, 20F), and the third coil body 30 but not to fix the core shaft 40. The second fixation portion 60 may be configured to fix the second coil body (20, 20F) and the third coil body 30 but not to fix the core shaft 40 or the first coil body 10.

This disclosure has been made to solve at least part of the problems described above and can be achieved in the following aspects.

(2) The medical device according to the above aspect may be configured such that the fixation portion fixes the core shaft and the first coil body.

According to this configuration, the fixation portion fixes the core shaft and the first coil body, so that a torquability of the medical device can be improved.

(3) The medical device according to the above aspects may be configured such that the fixation portion is a first fixation portion, and the medical device further includes: a third coil body disposed outside the second coil body in the radial direction; and a second fixation portion that fixes the second coil body and the third coil body on a proximal end side with respect to the first fixation portion.

According to this configuration, the torquability of the medical device can be improved by the second fixation portion.

(4) The medical device according to the above aspects may be configured such that the second fixation portion fixes the first coil body and the second coil body.

According to this configuration, the second fixation portion fixes the first coil body and the second coil body, so that the torquability of the medical device can be improved.

(5) The medical device according to the above aspects may be configured such that the second fixation portion fixes the core shaft and the first coil body.

According to this configuration, the second fixation portion fixes the core shaft and the first coil body, so that the torquability of the medical device can be improved.

The medical device according to the above aspects may be configured such that one or two of the first coil body, the second coil body, and the third coil body are wound in a first winding direction, and the remaining coil body is wound in a second winding direction different from the first winding direction.

According to this configuration, the coil bodies are wound in different directions, so that the torquability can be improved regardless of the rotational direction of the medical device.

(7) The medical device according to the above aspects may be configured such that the second coil body has a bending rigidity higher than a bending rigidity of the first coil body.

According to this configuration, the second coil body has a higher bending rigidity, so that the folding of the medical device can be further prevented from extending toward the proximal end side of the medical device.

(8) An aspect of the disclosed embodiments is a medical device. The medical device includes: a core shaft; a first coil body that is arranged so as to surround a distal end portion of the core shaft; a second coil body that is disposed outside the first coil body in a radial direction and has a distal end located between a distal end and a proximal end of the first coil body in an axial direction of the core shaft; and a fixation portion that fixes a vicinity of the distal end of the second coil body and the first coil body, in which, on a portion between an outer periphery and an inner periphery of the second coil body, an axially directional position of a distal end of the fixation portion is located on a position identical to or on a proximal end side with respect to an axially directional position of the distal end of the second coil body. The core shaft has: a straight portion having an outer diameter substantially constant in the axial direction of the core shaft; and a tapered portion disposed on a proximal end side with respect to the straight portion and having an outer diameter increasing from a distal end side toward a proximal end side in the axial direction of the core shaft, and on the portion between the outer periphery and the inner periphery of the second coil body in the fixation portion, the axially directional position of the distal end of the fixation portion is located on a position identical to or on a proximal end side with respect to an axially directional position of a distal end of the tapered portion.

According to this configuration, the distal end of the fixation portion is located on a position identical to or on a proximal end side with respect to the distal end of the second coil body, so that the folding of the medical device can be prevented from extending toward the proximal end side. Also, the distal end of the fixation portion is located on a position identical to or on a proximal end side with respect to the distal end of the tapered portion, so that the folding of the medical device can be prevented from extending toward the proximal end side.

The disclosed embodiments can be embodied in various forms, e.g. a form of a guide wire, a guide wire production method, a catheter production method, an endoscope, or a dilator.

### DESCRIPTION OF REFERENCE NUMERALS

1 . . . Medical device
10 . . . First coil body
11 . . . Wire of first coil body
20 . . . Second coil body
21 . . . Wire of second coil body
22 . . . Inner periphery of second coil body
23 . . . Outer periphery of second coil body
24 . . . Distal end of second coil body
30 . . . Third coil body
31 . . . Wire of third coil body
40 . . . Core shaft
41 . . . Straight portion
42 . . . Tapered portion
43 . . . Large diameter portion
44 . . . Distal end of tapered portion
50 . . . First fixation portion
51 . . . Specific portion
52 . . . Distal end of first fixation portion
60 . . . Second fixation portion
70 . . . Third fixation portion
80 . . . Fourth fixation portion
100 . . . Distal end-side fixation portion
110 . . . Proximal end-side fixation portion
S . . . First winding direction
Z . . . Second winding direction

What is claimed is:

1. A medical device comprising:
a core shaft;
a first coil body that is arranged so as to surround a distal end portion of the core shaft;
a second coil body that is disposed outside of the first coil body in a radial direction wherein the second coil body comprises a distal end disposed between a distal end and a proximal end of the first coil body in an axial direction of the core shaft; and
a fixation portion that fixes a vicinity of the distal end of the second coil body and the first coil body, in which, on a portion between an outer periphery and an inner periphery of the second coil body, an axial-direction position of a distal end of the fixation portion is located on a position identical to or on a proximal end side with respect to an axial-direction position of the distal end of the second coil body.

2. The medical device according to claim 1, wherein
the fixation portion fixes the core shaft and the first coil body.

3. The medical device according to claim 2, wherein
the fixation portion is a first fixation portion, and
the medical device further comprises:
a tubular body disposed outside of the second coil body in the radial direction; and
a second fixation portion that fixes the second coil body and the tubular body on a proximal end side with respect to the first fixation portion.

4. The medical device according to claim 3, wherein
the second fixation portion fixes the first coil body and the second coil body.

5. The medical device according to claim 4, wherein
the second fixation portion fixes the core shaft and the first coil body.

6. The medical device according to claim 5, wherein
the tubular body is a third coil body, and
one or two of the first coil body, the second coil body, and the third coil body are wound in a first winding direction, and the remaining coil body is wound in a second winding direction, wherein the second winding direction is different from the first winding direction.

7. The medical device according to claim 6, wherein
the second coil body has a bending rigidity higher than a bending rigidity of the first coil body.

8. The medical device according to claim 1, wherein
the fixation portion is a first fixation portion, and
the medical device further comprises:
a tubular body disposed outside the second coil body in the radial direction; and
a second fixation portion that fixes the second coil body and the tubular body on a proximal end side with respect to the first fixation portion.

9. The medical device according to claim 8, wherein
the second fixation portion fixes the first coil body and the second coil body.

10. The medical device according to claim 9, wherein
the second fixation portion fixes the core shaft and the first coil body.

11. The medical device according to claim 10, wherein
the tubular body is a third coil body, and
one or two of the first coil body, the second coil body, and the third coil body are wound in a first winding direction, and the remaining coil body is wound in a second winding direction, wherein the second winding direction is different from the first winding direction.

12. The medical device according to claim 11, wherein
the second coil body has a bending rigidity higher than a bending rigidity of the first coil body.

13. The medical device according to claim 3, wherein
the tubular body is a third coil body, and
one or two of the first coil body, the second coil body, and the third coil body are wound in a first winding direction, and the remaining coil body is wound in a second winding direction, wherein the second winding direction is different from the first winding direction.

14. The medical device according to claim 4, wherein
the tubular body is a third coil body, and
one or two of the first coil body, the second coil body, and the third coil body are wound in a first winding direction, and the remaining coil body is wound in a second winding direction, wherein the second winding direction is different from the first winding direction.

15. The medical device according to claim 1, wherein
the second coil body has a bending rigidity higher than a bending rigidity of the first coil body.

16. The medical device according to claim 2, wherein
the second coil body has a bending rigidity higher than a bending rigidity of the first coil body.

17. The medical device according to claim 3, wherein
the second coil body has a bending rigidity higher than a bending rigidity of the first coil body.

18. The medical device according to claim 4, wherein
the second coil body has a bending rigidity higher than a bending rigidity of the first coil body.

19. The medical device according to claim 5, wherein
the second coil body has a bending rigidity higher than a bending rigidity of the first coil body.

20. A medical device comprising:
a core shaft;
a first coil body that is arranged so as to surround a distal end portion of the core shaft;
a second coil body that is disposed outside of the first coil body in a radial direction wherein the second coil body comprises a distal end disposed between a distal end and a proximal end of the first coil body in an axial direction of the core shaft; and
a fixation portion that fixes a vicinity of the distal end of the second coil body and the first coil body, in which, on a portion between an outer periphery and an inner periphery of the second coil body, an axial-direction position of a distal end of the fixation portion is located on a position identical to or on a proximal end side with respect to an axial-direction position of the distal end of the second coil body, wherein
the core shaft comprises a straight portion having an outer diameter substantially constant in the axial direction of the core shaft,
a tapered portion disposed on the proximal end side with respect to the straight portion and having an outer diameter increasing from a distal end side toward a proximal end side in the axial direction of the core shaft, and
on the portion between the outer periphery and the inner periphery of the second coil body in the fixation portion, the axial-direction position of the distal end of the fixation portion is located on a position identical to or on a proximal end side with respect to an axial-direction position of a distal end of the tapered portion.
